(19) United States
(12) Patent Application Publication
(10) Pub. No.: US 2022/0204437 A1
(43) Pub. Date: Jun. 30, 2022

(54) New Kaurane analogues, their preparation and therapeutically uses

(71) Applicant: KEY PHARMA BIOMEDICAL INC., Dongguan, Guangdong (CN)

(72) Inventor: WEN TAN, (US)

(21) Appl. No.: 17/607,867

(22) PCT Filed: May 12, 2020
(86) PCT No.: PCT/US2020/032414

§ 371 Date: Oct. 30, 2021

Related U.S. Application Data
(60) Provisional application No. 62/846,760, filed on May 13, 2019.

(51) Int. Cl. C07C 62/26; C07C 62/06

(52) U.S. Cl. CPC C07C 62/26; C07C 62/06

(57) ABSTRACT

Synthesis of novel kaurane analogues , their preparation, and their uses in cardiac protection against ischemia, hypertrophy and chemotherapy induced cardiomyopathy as well as cerebral ischemia.

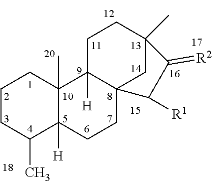

Wherein
R¹: formyl, carboxylic acid (carboxyl), salt of carboxylic acid
R²: oxygen, C-16 ketone to hydroxyl

### FIELD OF THE INVENTION:

The present invention relates to the synthesis of novel kaurane analogues , their preparation, and their uses in cardiac protection against ischemia, hypertrophy and chemotherapy induced cardiomyopathy. as well as cerebral ischemia.

### BACKGROUND OF THE INVENTION:

Kaurane compounds has been found pharmaceutically actives in protection against cardiac and cerebral ischemia, cardiac hypertrophy remodeling and cardiomyopathy (Patent:)

This invention disclosed new kuarane like compounds with similar pharmaceutically actives as previous disclosed. In addition, this invention disclosed these compounds were very effective in protection cardiac function against chemotherapy such as Doxorubicin.

It is known that several anti-cancer anthracyclines may induce severe acute or chronic cardiotoxicity, or even lethally heart failure. These anti-cancer drugs may manifest as an initial acute effect with transient tachycardia, or a delayed cardiomyopathy with cumulative dose- dependent congestive heart failure that persists longer period even after the drug treatment has been discontinued. Doxorubicin is an anthracycline cytostatic agent, have been widely used against broad range of cancers including several solid tumours and leukaemia. However, the cytotoxicity of Doxorubicin also damages to healthy tissues, especially to the cardiomyocyte.

One of the treatment options for chemotherapy drugs induced cardiomyopathy is to develop cardio-protective agents against the damage. The only approved prevention in the clinic for the Doxorubicin-induced cardiomyopathy is dexrazoxane. It inhibits the Doxorubicin cytotoxicity via. either blocking the iron-assisted oxidative radical production or inhibiting topoisomerase II A. However, dexrazoxane may attenuate the anti-cancer activity of Doxorubicin and the clinical usage of dexrazoxane has been hampered. Consequently, the development of novel protective agents against Doxorubicin-induced cardiomyopathy is needed.

Isosteviol (ent-16-ketobeyeran-19-oic acid is a tetracyclic diterpenoid having a beyerane skeleton, obtained by acid hydrolysis of Stevioside. Recently, Isosteviol has attaining growing interest of scientists because of it's unique structure, considerable pharmacological activities. It has been disclosed that evident that Isosteviol has shown a variety of biological activities such as cardio protective effect, anti-hypertension, anti-inflammatory, and alleviation of ischemia-reperfusion injury. This invention disclosed biological activities of new Isosteviol analogues, their synthesized and their protective efficiency against Doxorubicin-induced cardiotoxicity.

### DETAILS OF INVENTION

The object of the invention is to provide a simple synthetic method to obtain novel kauranes compounds. It is also an object of the invention to exhibit kaurene compounds in protecting activity against cardiac and cerebral ischemia, hypertrophy and cardiomyopathy and chemotherapy induced cardiac toxicity.

According to the invention, the said kaurene compounds have a general structure as in formula (I):

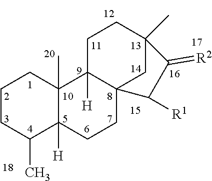

Wherein
R¹: formyl, carboxylic acid, salt of carboxylic acid
R²: C-16 ketone to hydroxyl

Synthesized novel analogues of kaurane, modifications at C-15 and C-16 positions resulted a potential application. All the synthesized new analogues of kauranes have shown very good protective activity against drugs induced cardiotoxicity.

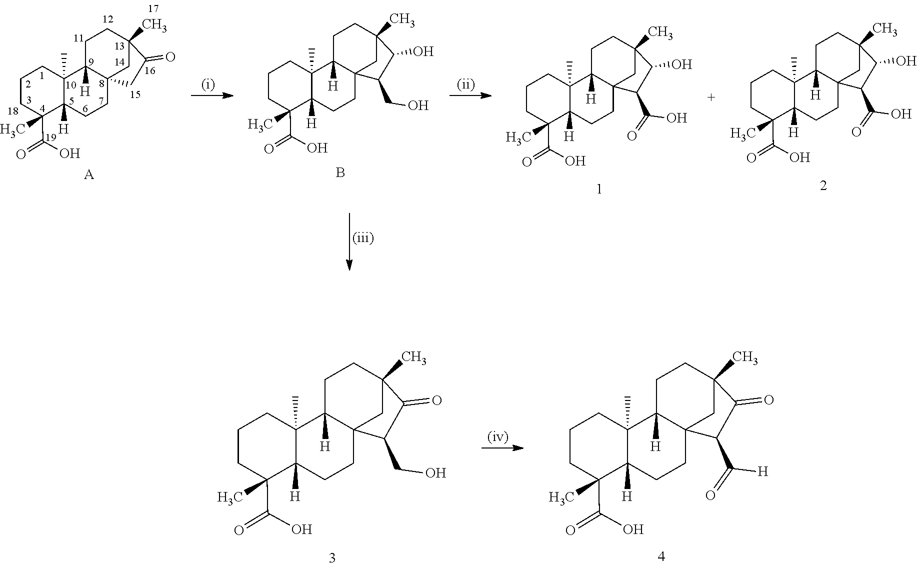

Reagents and conditions: (i) HCHO, NaOH, EtOH, 65 C; (ii) NaOCl, NaClO₂, TEMPO, KBr, ACN, 10 C; (iii) NaOCl, AcOH, rt; (iv) NaOCl, NaClO₂, TEMPO, KBr, ACN, 10 C.

Pharmacological Activity

This invention disclosed that among the synthesized new kaurane analogues, compounds 1 and 2 have shown maximum protective activity against Doxorubicin-induced cardiotoxicity. Further, we have showed that the synthesized compounds (1-4) co-treatment prevented Doxorubicin-induced cardiac dysfunctions in vivo.

TABLE 1
The LC₅₀ of DOX after co-treated with compounds of formula 1

| | | 95% Confidence |
|---|---|---|
| Group | LC₅₀/(μM) | interval/(μM) |
| DOX | 51.56 | 42.49-62.58 |
| DOX + 1 | 66.62 | 58.04-76.46 |
| DOX + 2 | 92.61 | 72.27-118.70 |
| DOX + 3 | 74.83 | 60.38-92.73 |
| DOX + 4 | 83.04 | 76.91-89.67 |

This invention disclosed that in cardiac toxicity animal induced by other drugs used for chemotherapy, such as anthracyclines, including, epirubicin, daunorubicin and aclarubicin, treatments of compound 1 and 2 showed Signiant beneficial effects in term of function and pathology is a widely used chemotherapy medicine with cardiotoxicity.

This invention also disclosed that in cardiac ischemia animal model by coronary ligation, compound 1 and compound 2 significantly increased the cardiac contractility and reduced the area of infraction and increased number of micro-blood vessels in comparison of control. In addition, compound 1 and 2 also reduced the occurrence of arrhythmia induced by acute ischemia. This invention disclosed that compound 1 and 2 can increased the function of failing heart due to acute coronary ligation.

Furthermore, in an embodiment of this invention, a chronic cardiac hypertrophy and heart failure animal was induced by TAC (Thoracic aorta constriction) . The animal was treated with compound 1 and 2 for consecutive 4 weeks after the surgery. The cardiac contractility was significantly enhanced in treated group in comparing of control animals. In histo- pathology, there were significant reduction of size of cardiomyocytes, reduced number of myofibroblasts, reduced interstitial collagen deposition in treatment animals in comparing to control.

In other embodiment, this invention disclosed that in a drug induced diabetes animal model, cardiomyopathy was developed after 4 weeks. However, the cardiac inflammatory changes were diminished after treatment of compound 1 and 2 for 2 weeks.

This invention disclosed that compound 1 and 2 can be used for treatment of ischemic heart diseases, heart failure, hypertrophy cardiac remolding and cardiomyopathy and arrhythmia.

This invention disclosed that in a cerebral ischemia animal model, treatments of compound 1 and 2 significantly reduced the area of infraction and improved the movement and behavers in comparison of control.

This invention disclosed that other compounds of formula 1 also showed similar beneficial effects as noted above.

### EXAMPLES

### Example 1 ,

Synthesis of compounds 1 and 2

To a stirred solution of compound B (500 mg, 1.42 mmol) in acetonitrile (25 mL), added NaClO₂ (1.42 mmol), KBr (30 mol%) and NaOCl (1.42 mmol) at 10° C. Finally charged TEMPO (10 mol%) and allowed to stir the reaction mixture for 1 h. Monitored the reaction progress using thin layer chromatography. Upon completion added water (15 mL) and adjusted the pH of the reaction medium to neutral using dilute HCl(0.01M). Extracted the products in to organic layer (ethylacetate), dried and removed the solvent by distillation under vacuum. The crude products (both acid and aldehyde derivatives) were purified by column chromatography (silica gel-100-200 mesh) eluted with ethylacetate/n-hexane.

Synthesis of compound 3

Charged NaOCl (1.14 mmol) in to a stirring solution of compound B (400 mg, 1.14 mmol) in acetic acid (7 mL) at room temperature. Monitored the reaction progress using thin layer chromatography. Upon completion, quenched the reaction by adding water. Extracted the product in to organic layer (ethylacetate), dried and removed the solvent by rotary evaporation under vacuum. The crude product was purified by column chromatography (silica gel-100-200 mesh) eluted with ethylacetate/n-hexane.

Synthesis of compound 4

To a stirred solution of compound 3 (300 mg, 0.86 mmol)in acetonitrile (10 mL), added NaClO₂ (0.86 mmol), KBr (30 mol%) and NaOCl (0.86 mmol) at 10° C. Finally charged TEMPO (10 mol%) and allowed to stir the reaction mixture for 1 h. Monitored the reaction progress using thin layer chromatography. Upon completion added water (15 mL) and adjusted the pH of the reaction medium to neutral using dilute HCl (0.01 M). Extracted the products in to organic layer (ethylacetate), dried in sodium sulphate and removed the solvent by distillation under vacuum. The crude products (both acid and aldehyde derivatives) were purified by column chromatography (silica gel-100-200 mesh) eluted with ethylacetate/n-hexane.

### Examples 2

Structure analysis

(4R, 4aS, 7S, 8R, 9S, 11aS, 11lbS)-8-hydroxy-4, 9, 11b-trimethyltetradecahydro-6a, 9-methanocyclohepta[a]naphthalene-4,7-dicarboxylic acid (1)

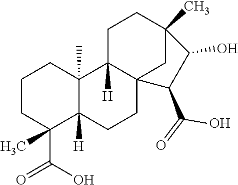

Yield: 312 mg, 60%; ¹H NMR δ _{H} (400 MHz, MeOD) 4.11 (1 H, d, J5.0), 2.90 (1 H, d, J3.8), 2.08 (2 H, dd, J21.6, 14.5), 1.92 (1 H, dd, J27.7, 13.9), 1.83-1.55 (7 H, m), 1.39 (1 H, d, J14.2), 1.29 (1 H, s), 1.24-1.19 (1 H, m), 1.17 (3 H, s), 1.13-1.04 (2 H, m), 1.02 (1 H, d, J4.1), 0.99 (3 H, s), 0.96 (1 H, s), 0.93 (3 H, s), 0.89 (1 H, d, J4.3); ¹³C NMR δ _{c} (100 MHz, MeOD) 181.63, 179.73, 84.33, 58.88, 58.45, 57.00, 55.33, 46.43, 44.59, 42.22, 40.94, 39.45, 39.17, 37.74, 34.22, 29.55, 25.16, 22.70, 20.65, 20.07, 13.25; HRMS (ESI) exact calculated mass for [M-1] (C₂₁H₃₂O₅) requires m/z 363.2249, found m/z 363.2208.

(4R, 4aS, 7S, 8R, 9S, 11aS, 11bS)-7-formyl-8-hydroxy-4, 9, llb-trimethyltetradecahydro-6a, 9-methanocyclohepta[a]naphthalene-4-carboxylic acid (2)

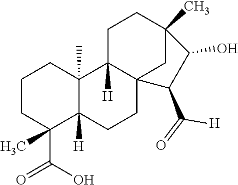

Yield: 149mg, 60%; ¹H NMR δ _{H} (400 MHz, CDCl₃) 9.93 (1 H, d, J2.6), 4.23 (1 H, d, J4.8), 2.93-2.86 (1 H, m), 2.15 (1 H, d, J 13.4), 1.94 (1 H, dt, J 13.9, 3.6), 1.80 (4 H, dd, J 15.0, 12.1), 1.43-1.35 (2 H, m), 1.23 (4 H, d, J2.6), 1.22(1 H, s), 1.18(1 H, s), 1.15(1 H, d, J2.8), 1.05 (3 H, dd, J13.0, 4.3), 0.98 (3 H, s), 0.96 (3 H, s), 0.93 (1 H, d, J4.2), 0.90 (1 H, d, J4.3), 0.88 - 0.84 (1 H, m); ¹³C NMR δ _{c} (100 MHz, CDCl₃) 204.37, 183.82, 78.52, 61.92, 57.39, 56.86, 54.02, 46.63, 43.71, 41.34, 39.52, 38.68, 37.82, 35.91, 33.13, 28.88, 24.69, 21.65, 19.75, 18.79, 13.13; HRMS (ESI) exact calculated mass for [M-1] (C₂₁H₃₂O₄) requires m/z 347.2300, found m/z 363.2331.

(4R, 4aS, 7R, 9S, 11aS, 11bS)-7-(hydroxymethyl)-4, 9, 11b-trimethyl-8-oxotetradecahydro-6a, 9-methanocyclohepta[a]naphthalene-4-carboxylic acid (3)

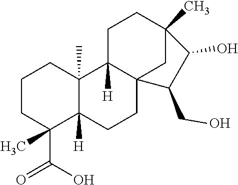

Yield: 354 mg, 89%; ¹H NMR δ _{H} (400 MHz, CDC1₃) 3.92 (1 H, dd, 10.7, 5.4), 3.68 (1 H, dd, J10.5, 9.1), 2.56-2.50 (1 H, m), 2.18 (1 H, d, J 13.4), 1.89(1 H, dd, J8.7, 4.7), 1.85-1.78 (2 H, m), 1.77-1.69 (3 H, m), 1.67-1.61 (1 H, m), 1.44 (2 H, dd, J12.8, 5.5), 1.36-1.29 (2 H, m), 1.26 (3 H, s), 1.23-1.16 (3 H, m), 1.05 (1 H, dd, J 13.5, 4.2), 0.99 (3 H, s), 0.98 -0.87 (2 H, m), 0.84 (3 H, s); ¹³C NMR δ _{c} (100 MHz, CDC1₃) 226.46, 183.44, 60.52, 57.26, 57.00, 53.04, 52.51, 48.66, 43.74, 40.72, 39.78, 38.59, 37.82, 37.19, 35.32, 29.14, 21.67, 19.94, 19.75, 18.94, 13.53; HRMS (ESI) exact calculated mass for [M+Na] (C₂₁H₃₂O₄) requires m/z 371.2198, found m/z 371.2131.

(4R, 4aS, 7S, 9S, 11aS, 11bS)-4, 9, 11b-trimethyl-8-oxotetradecahydro-6a, 9-methanocyclohepta[a]naphthalene-4, 7-dicarboxylic acid (4)

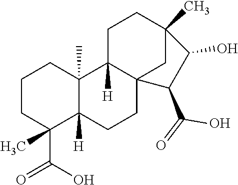

Yield: 232 mg, 78%; ¹H NMR δ _{H} (400 MHz, CDC1₃) 9.73 (1 H, d, J25.7), 2.68-2.62 (1 H, m), 2.18 (2 H, dd, J12.0, 2.2), 1.96(1 H, d, J13.6), 1.91-1.81 (2 H, m), 1.80-1.71 (3 H, m), 1.62-1.32(7 H, m), 1.28 (3 H, s), 1.20(1 H, d, J 11.5), 1.14(3 H, d, J6.2), 1.04(1 H, td, J13.6, 4.4), 0.94-0.85 (1 H, m), 0.63 (3 H, s); ¹³C NMR δ _{c} (101 MHz, CDC1₃) 209.72 (s), 188.52 (s), 183.69 (s), 79.61 (s), 59.01 (s), 57.61 (s), 52.75 (s), 50.16 (s), 48.99 (s), 48.57 (s), 43.68 (s), 40.32 (s), 38.67 (s), 38.60 (s), 37.49 (s), 29.11 (s), 21.93 (s), 21.28 (s), 20.29 (s), 19.20 (s), 15.77 (s); HRMS (ESI) exact calculated mass for [M-1] (C₂₁H₃₀O₄) requires m/z 345.2144, found m/z 345.2154.

1. kuarane analogue compounds of formula 1 of the following structures and its pharmaceutically acceptable salts. 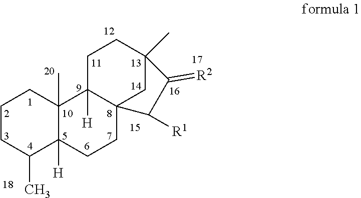
Wherein
R¹: formyl, carboxylic acid (carboxyl), salt of carboxylic acid
R²: oxygen, C-16 ketone to hydroxyl

2. A process producing compounds of claim 1.

3. A method of use compounds of claim 1 in manufactures of medicaments for treatment of cardiac diseases.

4. The said cardiac diseases in claim 3 are cardiac ischemia, cardiac hypertrophy, cardiomyopathy and heart failure.

5. The said cardiac diseases in claim 3 are cardiac toxicities induced by chemo-therapeutic medicine or other medicine.

6. The said chemo-therapeutic medicine said in claim 5 are anthracyclines including doxorubicin, epirubicin, daunorubicin and aclarubicin, 5, The said cardiac diseases in claim 3 is cardiac arrhythmia.

6. A method of use compounds of claim 1 in manufactures of medicaments for treatment of cerebral ischemia diseases.

7. The said of cerebral ischemia diseases in claim 6 are cerebral stroke and ischemic neuro-degenerative diseases.

8. A method of claim 3,wherein, the said medicaments are pharmaceutical preparations including: solid dose forms, gel, suppository, liquid or lyophilizes powder for injections, ointment or patches for topic use and aerosol or dry powders for lung inhalation or nasal.

9. The method of claim 1, wherein, the said pharmaceutical acceptable salts are hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.
